(19) United States
(12) Patent Application Publication
(10) Pub. No.: US 2024/0055097 A1
(43) Pub. Date: Feb. 15, 2024

(54) HEALTH RISK REDUCTION METHOD, HEALTH RISK REDUCTION SYSTEM, AND HEALTH RISK REDUCTION PROGRAM

(71) Applicant: Shimadzu Corporation, Kyoto-shi (JP)

(72) Inventor: Daisuke KAGAMI, Kyoto-shi (JP)

(21) Appl. No.: 18/335,446

(22) Filed: Jun. 15, 2023

(30) Foreign Application Priority Data
Aug. 10, 2022 (JP) 2022-127564

(51) Int. Cl. G16H 20/30; G16H 10/60

(52) U.S. Cl. CPC G16H 20/30; G16H 10/60

(57) ABSTRACT

A health risk reduction method includes a step of acquiring physical fitness test data by a physical fitness measurement performed to measure physical fitness of a subject, a step of acquiring cognitive function test data by a cognitive function test of the subject, a step of acquiring exercise content information to be proposed to the subject, based on the physical fitness test data and the cognitive function test data, and a step of outputting the exercise content information to be proposed to the subject.

### CROSS-REFERENCE TO RELATED APPLICATIONS

The priority application number JP2022-127564, entitled “Health Risk Reduction Method, Health Risk Reduction System, and Health Risk Reduction Program” filed on Aug. 10, 2022, KAGAMI Daisuke, upon which this patent application is based is hereby incorporated by reference in its entirety.

### BACKGROUND OF THE INVENTION

### Field of the Invention

The present invention relates to a health risk reduction method, a health risk reduction system, and a health risk reduction program for proposing exercises for maintaining the health of the elderly or the like.

### Description of the Background Art

Conventionally, there is known a health risk reduction system that proposes exercises for maintaining the health of the elderly or the like. Such a health risk reduction system is disclosed, for example, in Japanese Unexamined Patent Application Publication No. 2004-113471.

Japanese Unexamined Patent Application Publication No. 2004-113471 discloses a system provided with a chronological age input storage means for inputting and recording a chronological age of an individual, a physical fitness measuring means for measuring the physical fitness of the individual, a recording means for recording the physical fitness test data on a long-term basis, and an exercise prescription instruction means for instructing an exercise prescription for each individual. By comparing the chronological age of the individual with the physical fitness test data, the system can instruct an exercise prescription for each individual according to the chronological age.

Although not described in Japanese Unexamined Patent Application Publication No. 2004-113471, there is a correlation between cognitive functions and physical functions. Specifically, as physical functions decrease, the risk of a decrease in cognitive functions increases. Therefore, it is suggested that the improvement of physical functions by appropriate exercises based on the result of the physical fitness measurement leads to the improvement of cognitive functions.

In general, as age increases, the rate of dementia increases. Once dementia is developed, it is difficult to improve the symptoms. However, at the stage of mild cognitive impairment (MCI), which is a state before dementia, the onset of dementia can be prevented or delayed by taking appropriate measures.

However, there is a problem that the facility staff of the elderly facility, such as, e.g., a day service facility, could not have proposed exercises for preventing or delaying dementia to the user having an MCI risk of the elderly facility, such as, e.g., a day service facility.

### SUMMARY OF THE INVENTION

The present invention has been made to solve the above-described problems. It is an object of the present invention to provide a health risk reduction method, a health risk reduction system, and a health risk reduction program capable of proposing exercises for preventing or delaying dementia.

In order to achieve the above-described object, a health risk reduction method according to a first aspect of the present invention includes:
a step of acquiring physical fitness test data by a physical fitness measurement performed to measure physical fitness of a subject;
a step of acquiring cognitive function test data by a cognitive function test of the subject;
a step of acquiring exercise content information to be proposed to the subject, based on the physical fitness test data and the cognitive function test data; and
a step of outputting the exercise content information to be proposed to the subject.

A health risk reduction system according to a second aspect of the present invention includes:
a physical fitness test data acquisition unit configured to acquire physical fitness test data by a physical fitness measurement performed to measure physical fitness of a subject;
a test data acquisition unit configured to acquire cognitive function test data by a cognitive function test of the subject;
an exercise content information acquisition unit configured to acquire exercise content information to be proposed to the subject, based on the physical fitness test data and the cognitive function test data; and
an output unit configured to output the exercise content information to be proposed to the subject.

A health risk reduction program according to a third aspect of the present invention is configured to make a computer
control to acquire physical fitness test data by a physical fitness measurement performed to measure physical fitness of a subject,
control to acquire cognitive function test data by a cognitive function test of the subject;
control to acquire exercise content information to be proposed to the subject, based on the physical fitness test data and the cognitive function test data; and
control to output the exercise content information to be proposed to the subject.

As described above, the health risk reduction method according to the first aspect of the present invention includes a step of acquiring the exercise content information to be proposed to the subject, based on the physical fitness test data and the cognitive function test data, and a step of outputting the exercise content information to be proposed to the subject.

With this, when acquiring the physical fitness test data and the cognitive function test data of the subject, by acquiring the exercise content information for preventing or delaying dementia according to the subject, based on the physical fitness test data and the cognitive function test data of each subject, it is possible to provide a health risk reduction method capable of proposing exercises for preventing or delaying dementia.

As described above, the health risk reduction system according to the second aspect of the present invention includes an exercise content information acquisition unit configured to acquire exercise content information to be proposed to the subject, based on the physical fitness test data and the cognitive function test data, and an output unit configured to output the exercise content information to be proposed to the subject.

With this, as in the health risk reduction method according to the first aspect of the present invention, when acquiring the physical fitness test data and the cognitive function test data of the subject, by acquiring the exercise content information for preventing or delaying dementia according to the subject, based on the physical fitness test data and the cognitive function test data of each subject, it is possible to provide a health risk reduction system capable of proposing exercises for preventing or delaying dementia.

As described above, the health risk reduction program according to the third aspect of the present invention is configured to make a computer control to acquire exercise content information to be proposed to the subject based on the physical fitness test data and the cognitive function test data, and control to output the exercise content information to be proposed to the subject.

With this, as in the first and second aspects of the present invention, when acquiring the physical fitness test data and the cognitive function test data of the subject, by acquiring the exercise content information for preventing or delaying dementia according to the subject, based on the physical fitness test data and the cognitive function test data of each subject, it is possible to provide a health risk reduction program capable of proposing exercises for preventing or delaying dementia

### BRIEF DESCRIPTION OF THE DRAWINGS

FIG. 1 is a block diagram showing an entire configuration of a health risk reduction system according to one embodiment of the present invention.

FIG. 2 is a block diagram for showing an exemplary configuration of a facility staff terminal according to one embodiment of the present invention.

FIG. 3 is a block diagram for showing an exemplary configuration of a server according to one embodiment of the present invention.

FIG. 4 is a screen image displayed on a facility staff terminal according to one embodiment of the present invention.

FIG. 5 is a diagram showing exercise content information and a ratio of physical fitness test data acquired based on MCI risk data according to this embodiment.

FIG. 6 is a flowchart showing display processing of exercise content information by a health risk reduction system.

### DESCRIPTION OF THE PREFERRED EMBODIMENTS

Hereinafter, some embodiments in which the present invention is embodied will be described with reference to the attached drawings.

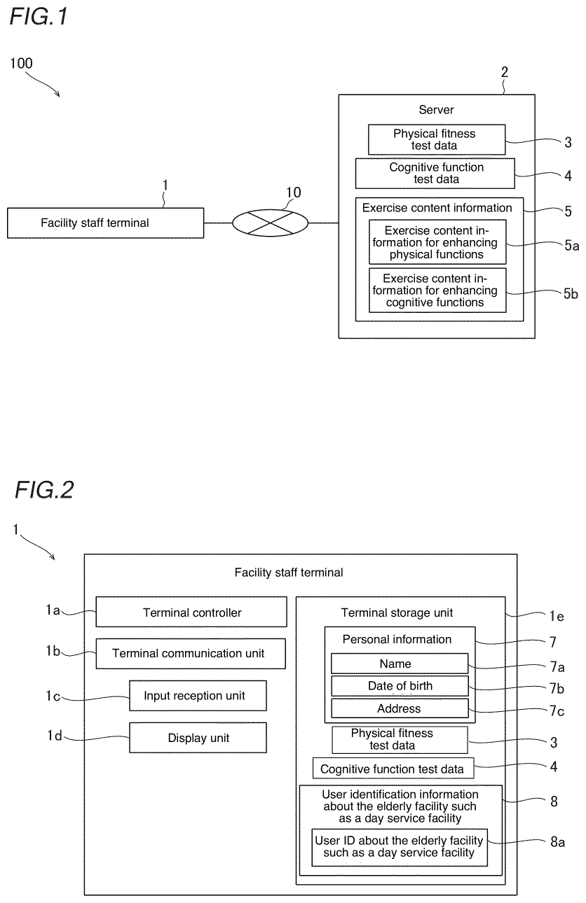

The configuration of a health risk reduction system 100 according to this embodiment will be described with reference to FIG. 1 to FIG. 3.

The health risk reduction system 100 is configured to propose exercise contents for preventing the onset of dementia or delaying dementia to a facility staff of an elderly facility, such as, e.g., a day service facility. Note that the facility staff makes the user of the elderly facility, such as, e.g., a day service facility, perform exercise contents, based on the exercise contents proposed by the health risk reduction system 100.

### (Entire Configuration of Health Risk Reduction System)

As shown in FIG. 1, the health risk reduction system 100 is provided with a facility staff terminal 1 and a server 2.

As shown in FIG. 1, the facility staff terminal 1 and the server 2 are connected via a network 10. The network 10 is an open network that can be connected from devices other than the facility staff terminal 1.

The facility staff terminal 1 is configured to register (store), in the server 2 via the network 10, the information on the input physical fitness test data 3 of the user of the elderly facility, such as, e.g., a day service facility, and the input cognitive function test data 4.

The facility staff terminal 1 includes, for example, a smart phone, a tablet terminal, and the like. The detailed configuration of the facility staff terminal 1 will be described later.

Note that the physical fitness test data 3 include information generated based on physical fitness measurement results of a user of an elderly facility, such as, e.g., a day service facility. For example, in a case where physical fitness measurement results are the generation average or above (the physical fitness age is average or above), the physical fitness test data 3 include information A. In a case where physical fitness measurement results are the generation average or below (physical fitness age is average or below), the physical fitness test data 3 include information B.

The cognitive function test data 4 includes an MCI result (MCI risk data) of an MCI screening test acquired by a blood test.

The MCI risk data is represented by a numerical value in the range of 0.00 to 2.00. The higher the number, the higher the MCI risk. For example, when the MCI risk data is within the range of 0.00 and 0.50, it indicates A in which the MCI risk is almost none. When the MCI risk data is within the range of 0.51 and 1.00, it indicates B in which the MCI risk is low. When the MCI risk data is within the range of 1.01 to 1.50, it indicates C in which the MCI risk is medium. When the MCI risk data is within the range of 1.51 to 2.00, it indicates D in which the MCI risk is high. A user of an elderly facility, such as, e.g., a day service facility, is one example of the “subject” recited in claims.

The server 2 stores exercise content information 5a for enhancing physical functions and exercise content information 5b for enhancing cognitive functions, both of which being exercise content information 5. The server 2 stores (registers) information on the physical fitness test data 3 and the cognitive function test data 4 of the user of the elderly facility, such as, e.g., a day service facility, which are input through the facility staff terminal 1 by a facility staff.

Further, the server 2 selects exercise content information 5 based on the registered (stored) physical fitness test data 3 and cognitive function test data 4, and outputs the exercise content information 5 to the facility staff terminal 1. The detailed configuration of the server 2 will be described later.

### (Facility Staff Terminal)

As shown in FIG. 2, the facility staff terminal 1 is provided with a terminal controller 1a, a terminal communication unit 1b, an input reception unit 1c, a display unit 1d, and a terminal storage unit 1e. The facility staff terminal 1 is configured to be capable of inputting personal information 7 of the user of the elderly facility, such as, e.g., a day service facility, physical fitness test data 3 which are information other than the personal information 7, cognitive function test data 4, and user identification information 8 on the elderly facility, such as, e.g., a day service facility, the user identification information being different from the personal information 7.

In this embodiment, the facility staff inputs the personal information 7 and the user identification information 8 on the elderly facility, such as, e.g., a day service facility, by activating a data input application (not shown) by the facility staff terminal 1 or by accessing a data input service (not shown) and inputting data.

The personal information 7 includes the name 7a, the date of birth 7b, and the address 7c of the user of the elderly facility, such as, e.g., a day service facility. Further, the user identification information 8 on the elderly facility, such as, e.g., a day service facility, includes a user ID 8a of the elderly facility, such as, e.g., a day service facility.

The user ID 8a of the elderly facility, such as, e.g., a day service facility, is information not including the name 7a, the date of birth 7b, and the address 7c of the user of the elderly facility, such as, e.g., a day service facility. Specifically, the user ID 8a of the elderly facility, such as, e.g., a day service facility, is configured by a character string unique to the user of the elderly facility, such as, e.g., a day service facility, which is given to identify the user of the elderly facility, such as, e.g., a day service facility, in the server 2.

The terminal controller 1a is configured to control the facility staff terminal 1. The terminal controller 1a includes, for example, a processor, such as, e.g., a CPU (Central Processing Unit), and a memory, such as, e.g., a ROM (Read Only Memory) and a RAM

### (Random Access Memory).

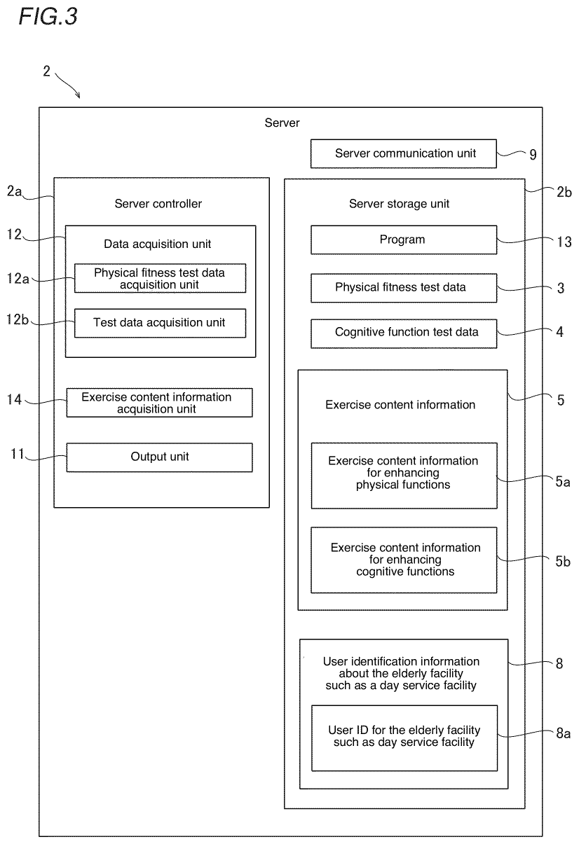

The terminal communication unit 1b is configured to be connectable to the server 2 via the network 10. The terminal communication unit 1b is configured to transmit the input physical fitness test data 3 and cognitive function test data 4 to a server storage unit 2b (see FIG. 3) in a data input application.

Specifically, the facility staff terminal 1 transmits the physical fitness test data 3 and the cognitive function test data 4 input to the input reception unit 1c by the facility staff to the server communication unit 9 via the network 10. The terminal communication unit 1b includes an interface for wired communication, an interface for radio communication, and the like.

The input reception unit 1c is configured to be accept an operation input from a facility staff. The input reception unit 1c includes, for example, an input device, such as, e.g., a touchpad and a keyboard.

The display unit 1d is configured to display, for example, the contents of physical fitness test data 3 and cognitive function test data 4 when a facility staff inputs the physical fitness test data 3 and the cognitive function test data 4. Further, the display unit 1d displays exercise content information 5 received from the server 2. The display unit 1d includes, for example, a liquid crystal monitor. Note that the input reception unit 1c and the display unit 1d may be integrally formed like a touch panel, for example.

The terminal storage unit 1e is configured to, when the facility staff inputs the personal information 7, the physical fitness test data 3, and the cognitive function test data 4 of the user of the elderly facility, such as, e.g., the day service facility, store the user identification information 8 of the elderly facility, such as, e.g., a day service facility, the personal information 7, the physical fitness test data 3, and the cognitive function test data 4, which are received from the server 2, in association with each other. The terminal storage unit 1e includes, for example, a flash memory.

### (Server)

The server 2 stores the physical fitness test data 3 and the cognitive function test data 4. Further, the server 2 selects the exercise content information 5 from a plurality of pieces of exercise content information 5 set in advance, based on the analysis result of the physical fitness test data 3 and the analysis result of the cognitive function test data 4. Further, the server 2 outputs the selected exercise content information 5 to be proposed to the user of the elderly facility, such as, e.g., a day service facility, to the terminal.

As shown in FIG. 3, the server 2 includes a server controller 2a, a server storage unit 2b, and a server communication unit 9. The server 2 receives the physical fitness test data 3 and the cognitive function test data 4 input to the facility staff terminal 1. The server 2 is configured to store the physical fitness test data 3, the cognitive function test data 4, and the user identification information 8 about the elderly facility, such as, e.g., a day service facility, which were transmitted via the network 10, in association with each other.

The server controller 2a is configured to control the server 2. The server controller 2a includes, for example, a CPU, a RAM, and a ROM.

The server controller 2a causes the physical fitness test data 3 and the cognitive function test data 4 received from the facility staff terminal 1 via the network 10 to store in the server storage unit 2b.

The server controller 2a includes an output unit 11, a data acquisition unit 12, and an exercise content information acquisition unit 14. The server controller 2a functions as the output unit 11, the data acquisition unit 12, and the exercise content information acquisition unit 14 by executing programs 13 stored in the server storage unit 2b.

The output unit 11 is configured to output the exercise content information 5 to be proposed to the user of the elderly facility, such as, e.g., a day service facility, to the facility staff terminal 1 to the server communication unit 9. The output unit 11 is, for example, a CPU.

The data acquisition unit 12 includes a physical fitness test data acquisition unit 12a for acquiring the physical fitness test data 3 and a test data acquisition unit 12b for acquiring the cognitive function test data 4. The physical fitness test data acquisition unit 12a acquires the physical fitness test data 3 input to the facility staff terminal 1 via the network 10. The test data acquisition unit 12b acquires the cognitive function test data 4 input to the facility staff terminal 1 via the network 10. The exercise content information acquisition unit 14 acquires, based on the physical fitness test data 3 and the cognitive function test data 4, the exercise content information 5 from the exercise content information 5a for enhancing physical functions and the exercise content information 5b for enhancing cognitive functions stored in the server storage unit 2b.

The server storage unit 2b is configured to store programs 13, physical fitness test data 3, cognitive function test data 4, and a user ID 8a of the elderly facility, such as, e.g., a day service facility, of the user identification information 8 about the elderly facility, such as, e.g., a day service facility.

Further, the server storage unit 2b stores programs 13 executed by the server controller 2a and exercise content information 5. The exercise content information 5 to be proposed to the user of the elderly facility, such as, e.g., a day service facility, is acquired based on the physical fitness test data 3 and the cognitive function test data 4. The server storage unit 2b stores the exercise content information 5a for enhancing physical functions and the exercise content information 5b for enhancing cognitive functions, which are the exercise content information 5. The program 13 is a computer program executed by the server 2. The server storage unit 2b includes, for example, an HDD, or an SSD.

The server communication unit 9 is configured to be connectable to the facility staff terminal 1 via the network 10. The server communication unit 9 includes an interface for wired communication, an interface for radio communication, and the like.

### (Exercise Content Information)

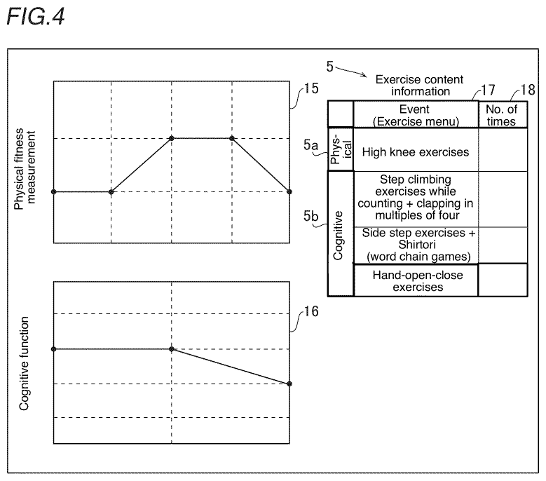

The exercise content information 5 of this embodiment includes events (exercise menu) 17 and the number of times 18, as shown in FIG. 4. The exercise content information 5 includes two types of information, i.e., exercise content information 5a for enhancing physical functions, and exercise content information 5b for enhancing cognitive functions. The events (exercise menu) 17 of the exercise content information 5a for enhancing physical functions provide a muscle training menu including, e.g., high knee exercises, abdominal muscle exercises, and back muscle exercises.

Further, the events (exercise menu) 17 of the exercise content information 5b for enhancing cognitive functions are a combination of an action for enhancing cognitive functions and a physical exercise. The action includes, for example, clapping in multiples of four by simultaneously performing step climbing exercises while counting, performing a shiritori game (Japanese word chain game) while performing side step exercises, and performing palm-open-close exercise according to a rhythm.

### (Flow up to Displaying Exercise Content Information)

In this embodiment, physical fitness test data 3 by a physical fitness measurement performed to measure the physical fitness of the user of the elderly facility, such as, e.g., a day service facility, is acquired. When the facility staff inputs the fitness measurement results performed by the user of the elderly facility, such as, e.g., a day service facility, at the day service facility to the facility staff terminal 1, as the physical fitness test data 3, the server 2 acquires the physical fitness test data 3.

The server 2 acquires the cognitive function test data 4 by the cognitive function test of the user of the elderly facility, such as, e.g., a day service facility. The server 2 acquires the cognitive function test data 4 when the facility staff inputs, as the cognitive function test data 4 to the facility staff terminal 1, the results of a screening test, such as, e.g., a blood test of the user of the elderly facility, such as, e.g., a day service, performed at a medical institution.

Further, the acquired physical fitness test data 3 and the measurement time point are stored in association with each other. Similarly, the acquired cognitive function test data 4 and the test time point are stored in association with each other. The measurement time point indicates the date on which the physical fitness measurement of the day service facility user was performed. The test time point indicates the test date on which the screening test of the user of the elderly facility, such as, e.g., a day service facility, was performed.

The physical fitness test data 3 and the cognitive function test data 4 acquired as described above are input to the facility staff terminal 1 to be registered (stored) in the server storage unit 2b. The exercise content information acquisition unit 14 acquires the exercise content information 5 to be proposed to the user of the elderly facility, such as, e.g., a day service facility, based on the physical fitness test data 3 and the cognitive function test data 4. The exercise content information acquisition unit 14 acquires, as exercise content information 5, the exercise content information 5a for enhancing physical functions and the exercise content information 5b for enhancing cognitive functions, which were stored in the server storage unit 2b.

At this time, the server 2 analyzes the physical fitness test data 3 and the cognitive function test data 4 to select the exercise content information 5 from the plurality of pieces of the exercise content information 5 set in advance, based on the analysis result of the physical fitness test data 3 and the analysis result of the cognitive function test data 4.

Further, the server 2 differentiates, when acquiring the exercise content information 5, the ratio of the exercise for enhancing physical functions and the ratio of the exercise for enhancing cognitive functions, according to the analysis result of the physical fitness test data 3 and the analysis result of the cognitive function test data 4.

In this embodiment, four pieces of exercise content information 5 are selected from a plurality of pieces of exercise content information 5 stored in the server 2. Based on the result of the physical fitness test data 3 and the result of the cognitive function test data 4, the exercise content information 5 is selected. For example, when the MCI risk data resulting from the cognitive function test data 4 is C (MCI risk is medium) or D (MCI risk is high), one exercise leading to improvement of physical functions and three exercises leading to improvement of cognitive functions are selected. Other specific examples will be described later.

When a user ID 8a of an elderly facility, such as, e.g., a day service facility, is input to the facility staff terminal 1 by the facility staff, the acquired exercise content information 5 is output to the facility staff terminal 1. When the exercise content information 5 is output, the facility staff terminal 1 displays the physical fitness test data 3 and the cognitive function test data 4 along the time series on the display unit 1d, in addition to the exercise content information 5.

Further, the facility staff terminal 1 displays the time series change of the physical fitness test data 3 within a predetermined period and the time series change of the cognitive function test data 4 within a predetermined period. As shown in FIG. 4 showing one example of a display screen, the display unit 1d displays, in addition to the exercise content information 5 to be proposed to the day service facility user, a graph 15 of the time series change of the physical fitness test data 3 and a graph 16 of the time series change of the cognitive function test data 4.

### (Acquisition of Exercise Content Information)

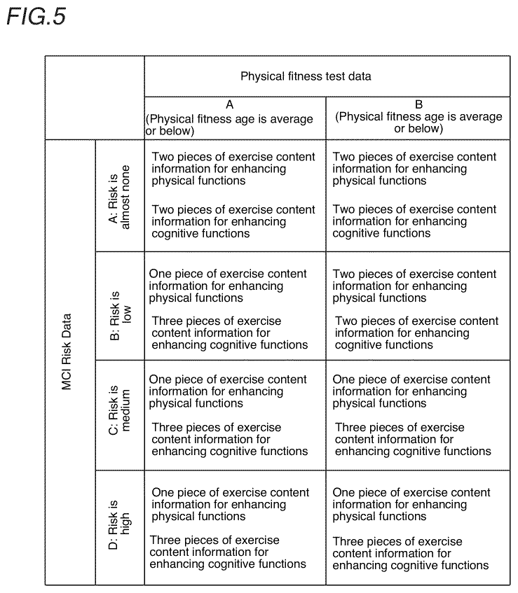

Referring to FIG. 5, the acquisition of the exercise content information 5 of the server 2 will be described. In this embodiment, the physical fitness test data 3 is classified into two types: A in which the physical fitness age is average or above, and B in which the physical fitness age is average or below. The MCI risk data is classified into A in which the MCI risk is almost none, B in which the MCI risk is low, C in which the MCI risk is medium, and D in which the MCI risk is high.

When the MCI risk is C or D, the server 2 proposes one piece of exercise content information 5a for enhancing physical functions and three pieces of exercise content information 5b for enhancing cognitive functions. When the MCI risk is B and the physical fitness age is A, the server 2 proposes one piece of exercise content information 5a for enhancing physical functions and three pieces of exercise content information 5b for enhancing cognitive functions. When the MCI risk is B and the physical fitness age is B, the server 2 proposes two pieces of exercise content information 5a for enhancing physical functions and two pieces of exercise content information 5b for enhancing cognitive functions. When the MCI risk is A, the server 2 proposes two pieces of exercise content information 5a for enhancing physical functions and two pieces of exercise content information 5b for enhancing cognitive functions.

### (Display Processing of Exercise Content Information)

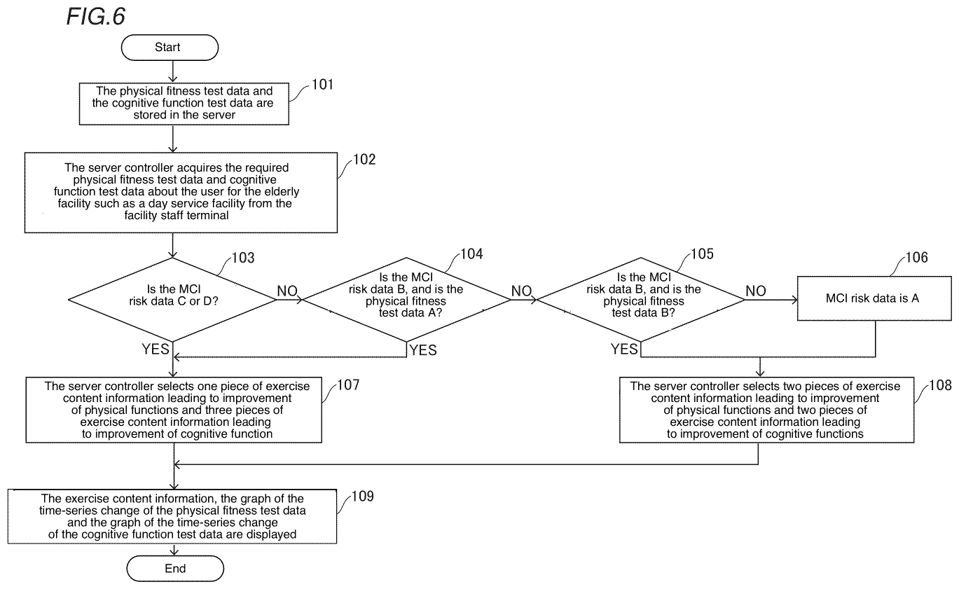

Next, referring to FIG. 6, the display processing of the exercise content information 5 by the health risk reduction system 100 of this embodiment will be described based on a flowchart.

First, in Step 101, the physical fitness test data 3 and the cognitive function test data 4 input to the facility staff terminal 1 are input. The input physical fitness test data 3 and cognitive function test data 4 are transmitted from the terminal communication unit 1b to the server communication unit 9 via the network 10. The server controller 2a registers (stores) the received physical fitness test data 3 and cognitive function test data 4 in the server storage unit 2b.

Then, in Step 102, the user ID 8a of the elderly facility, such as, e.g., a day service facility, is input to the facility staff terminal 1. The facility staff terminal 1 requests the exercise content information 5 of the user of the elderly facility, such as, e.g., a day service facility, to the server 2 when the user ID 8a of the elderly facility, such as, e.g., the day service facility is input. The data acquisition unit 12 acquires the requested physical fitness test data 3 and cognitive function test data 4 of the user of the elderly facility, such as, e.g., a day service facility, from the server storage unit 2b.

In Step 103, when the MCI risk data is not C (MCI risk is medium) or D (MCI risk is high), the server controller 2a advances the processing to Step 104. Further, in Step 103, when the MCI risk data is C (MCI risk is medium) or D (MCI risk is high), the server controller 2a advances the processing to Step 107.

In Step 104, when the MCI risk data is not B (MCI risk is low) and the physical fitness test data 3 is not A (physical fitness age is average or above), the server controller 2a advances the processing to Step 105. Further, in Step 104, when the MCI risk data is B (MCI risk is low) and the physical fitness test data 3 is A (physical fitness age is average or above), the server controller 2a advances the processing to Step 107.

In Step 105, when the MCI risk data is B (MCI risk is low) and the physical fitness test data 3 is not B (physical fitness age is average or less), the server controller 2a advances the processing to Step 106. Further, in Step 105, when the MCI risk data is B (MCI risk is low) and the physical fitness test data 3 is B (physical fitness age is average or below), the server controller 2a advances the processing to Step 108.

In Step 106, when the MCI risk data is A (MCI risk is almost none), the server controller 2a advances the processing to Step 108.

In Step 107, the server controller 2a selects one piece of exercise content information 5a leading to an improvement of physical functions, and three pieces of exercise content information 5b leading to an improvement of cognitive functions.

In Step 108, the server controller 2a selects two pieces of exercise content information 5a leading to the improvement of physical functions and two pieces of exercise content information 5b leading to the improvement of cognitive functions.

In Step 109, the server controller 2a transmits the exercise content information 5, the graph 15 of the time-series change of the physical fitness test data 3, and the graph 16 of the time-series change of the cognitive function test data 4 to the facility staff terminal 1 via the network 10. The facility staff terminal 1 displays the received exercise content information 5, graph 15 of the time-series change of the physical fitness test data 3, and graph 16 of the time-series change of the cognitive function test data 4 on the display unit 1d. With this, the processing of the server controller 2a ends.

### (Effects of This Embodiment)

In this embodiment, the following effects can be obtained.

This embodiment includes, as described above, a step of acquiring the physical fitness test data 3 by a physical fitness measurement performed to measure the physical fitness of the subject of the elderly facility, such as, e.g., a day service facility, a step of acquiring the cognitive function test data 4 by the cognitive function test of the subject of the elderly facility, such as, e.g., day service facility, a step of acquiring the exercise content information 5 to be proposed to the subject of the elderly facility, such as, e.g., a day service facility, based on the physical fitness test data 3 and the cognitive function test data 4, and a step of outputting the exercise content information 5 to be proposed to the subject of the elderly facility, such as, e.g., the day service.

With this, when acquiring the physical fitness test data 3 and the cognitive function test data 4 of the user of the elderly facility, such as, e.g., a day service facility, by acquiring the exercise content information 5 for preventing or delaying dementia according to the user of the elderly facility, such as, e.g., a day service facility, based on the physical fitness test data 3 and the cognitive function test data 4 of the individual user of the elderly facility, such as, e.g., a day service facility, it is possible to provide a health risk reduction method capable of suggesting exercises for preventing or delaying dementia.

Further, in this embodiment, the step of outputting the exercise content information 5 as described above includes a step of displaying the physical fitness test data 3 and the cognitive function test data 4, in addition to the exercise content information 5. This allows the facility staff to visually confirm the exercise content information 5, the physical fitness test data 3, and the cognitive function test data 4. Therefore, the facility staff can propose more suitable exercise contents to the user of the elderly facility, such as, e.g., a day service facility.

Further, in this embodiment, as described above, in the step of acquiring the exercise content information 5, the exercise content information 5a for enhancing cognitive functions and the exercise content information 5b for enhancing physical functions are acquired. As a result, the facility staff can acquire the exercise content information 5a for enhancing cognitive functions and the exercise content information 5b for enhancing physical functions. Therefore, the facility staff can make the user of the elderly facility, such as, e.g., a day service facility, exercise by selecting exercise contents from the acquired exercise content information 5 without considering all exercise contents for enhancing cognitive functions and exercise contents for enhancing physical functions.

Also, in this embodiment, as described above, the step of acquiring the exercise content information 5 includes a step of analyzing the physical fitness test data and the cognitive function test data 4, and a step of selecting the exercise content information 5 from a plurality of pieces of exercise content information 5 set in advance, based on the analysis result of the physical fitness test data 3 and the cognitive function test data 4. This eliminates the need for the facility staff to perform an analysis, from the physical fitness test data 3 and the cognitive function test data 4. Therefore, the burden on the facility staff can be reduced, and exercises suitable for the user of elderly facility, such as, e.g., a day service facility, can be proposed.

Also, in this embodiment, as described above, in the step of acquiring the exercise content information 5, the ratio of exercises for enhancing physical functions and the ratio of exercises for enhancing the cognitive functions are differentiated according to the analysis result of the physical fitness test data 3 and the analysis result of the cognitive function test data 4. With this, the facility staff can know the ratio of exercises for enhancing physical functions and the ratio of exercises for enhancing cognitive functions, both of which are required for the user of the elderly facility, such as, e.g., a day service, and can propose more appropriate exercises leading to dementia prevention of the user of the elderly facility, such as, e.g., a day service facility, by proposing more appropriate ratios of exercises.

Also, in this embodiment, as described above, the step of acquiring the physical fitness test data 3 includes a step of storing the acquired physical fitness test data 3 and the measurement time point in association with each other. The step of acquiring the cognitive function test data 4 includes a step of storing the acquired cognitive function test data 4 and the test time point in association with each other. In the display step, the physical fitness test data 3 and the cognitive function test data 4 within a predetermined period are displayed along the time series.

With this, the facility staff can visually confirm the physical fitness test data 3 and the cognitive function test data 4 of the user of the elderly facility, such as, e.g., a day service facility, along the time series. Therefore, it is possible to accurately determine the state of the user of the elderly facility, such as, e.g., a day service facility. The state of the user includes, for example, the change in the state from the past to the present. Therefore, the facility staff can propose exercise contents according to the state of the user of the elderly facility, such as, e.g., a day service facility.

Further, in this embodiment, as described above, in the display step, the time series change of the physical fitness test data 3 within the predetermined period and the time series change of the cognitive function test data 4 within the predetermined period are respectively displayed. This allows the facility staff to confirm the effects of physical functions and cognitive functions based on the exercises performed by the user of the elderly facility, such as, e.g., a day service facility, from the past to the present. Therefore, it is possible to make the user of the elderly facility, such as, e.g.,, a day service facility, selectively execute highly effective exercises executed in the past.

In addition, in this embodiment, as described above, in the display step, the graph 15 of the time series change of the physical fitness test data 3 within the predetermined period and the graph 16 of the time series change of the cognitive function test data 4 within the predetermined period are displayed side by side. With this, the facility staff can confirm the time series change by the graph, so that it is possible to easily find a time point at which physical functions were improved and a time point at which cognitive functions were improved.

In addition, in this embodiment, as described above, in the display step, in addition to the graph 15 of the time series change of the physical fitness test data 3 and the graph 16 of the time series change of the cognitive function test data 4, the exercise content information 5 to be proposed to the user of the elderly facility, such as, e.g., a day service facility, are displayed side by side. This allows the facility staff to easily find the time point at which physical functions were improved and the time point at which cognitive functions were improved, and also allows the facility user to confirm the appropriate exercise content information 5 for the user of the elderly facility, such as, e.g., a day service facility. Therefore, exercises leading to the improvement of physical functions and cognitive functions can be proposed.

In this embodiment, as described above, the exercise content information 5 includes the events 17 and the number of times 18. As a result, the facility staff can easily determine what kind of events 17 and how many times are appropriate for the user of the elderly facility, such as, e.g., a day service facility, so that it is possible to propose appropriate exercises for the user of the elderly facility.

In addition, in this embodiment, as described above, the health risk reduction system 100 includes: a physical fitness test data acquisition unit 12a configured to acquire physical fitness test data 3 according to a physical fitness measurement performed to measure the physical fitness of the subject of the elderly facility, such as, e.g., a daily service facility; the test data acquisition unit 12b configured to acquire cognitive function test data 4 according to the cognitive function test of the subject of the elderly facility, such as, e.g., a daily service facility; the exercise content information acquisition unit 14 configured to acquire the exercise content information 5 to be proposed to the subject of the elderly facility, such as, e.g., a daily service facility, based on the physical fitness test data 3 and the cognitive function test data 4; and the output unit 11 configured to output the exercise content information 5 to be proposed to the subject of the elderly facility, such as, e.g., a daily service facility.

With this, when acquiring the physical fitness test data 3 and the cognitive function test data 4 of the user of the elderly facility, such as, e.g., a day service facility, by acquiring the exercise content information 5 for preventing or delaying dementia according to the user of the elderly facility, such as, e.g., a day service, based on the physical fitness test data 3 and the cognitive function test data 4 of the user of the elderly facility, such as, e.g., a day service facility, it is possible to provide a health risk reduction system 100 capable of suggesting exercises for preventing or delaying dementia.

In addition, in this embodiment, as described above, a health risk reduction program configured to make a computer control to acquire physical fitness test data 3 by a physical fitness measurement performed to measure physical fitness of a user of the elderly facility, such as, e.g., a day service facility, control to acquire cognitive function test data 4 by a cognitive function test of the user of the elderly facility, such as, e.g., a day service facility, control to acquire exercise content information 5 to be proposed to the user of the elderly facility, such as, e.g., a day service facility, based on the physical fitness test data 3 and the cognitive function test data 4, and control to output the exercise content information 5 to be proposed to the subject.

With this, when acquiring the physical fitness test data 3 and the cognitive function test data 4 of the user of the elderly facility, such as, e.g., a day service facility, by acquiring the exercise content information 5 for preventing or delaying dementia according to the user of the elderly facility, such as, e.g., a day service, based on the physical fitness test data 3 and the cognitive function test data 4 of the user of the elderly facility, such as, e.g., a day service facility, it is possible to provide a health risk reduction program capable of suggesting exercises for preventing or delaying dementia.

### [Modified Embodiments]

It should be understood that the embodiments disclosed here are examples in all respects and are not restrictive. The scope of the present invention is shown by the scope of the claims rather than the descriptions of the embodiments described above, and includes all changes (modifications) within the meaning of equivalent and the scope of claims.

For example, in the above-described embodiment, an example is shown in which four pieces of exercise content information 5 are selected, but the present invention is not limited thereto. For example, it may be configured to acquire one to three or five or more pieces of exercise content information 5 according to the physical fitness of the user of the elderly facility, such as, e.g., a day service facility.

For example, in the above-described embodiment, an example is shown in which the evaluation criteria of the physical fitness test data 3 is selected from two types: A in which the result of the physical fitness measurement is average or above (physical fitness age is average or above), and B in which the result of the physical fitness measurement is generation average or below (physical fitness age is average or below), but the present invention is not limited thereto. Three or more types may be used. For example, the physical fitness test data 3 of the user of the elderly facility, such as, e.g., a day service facility, may be expressed as “physical fitness age in the 40s to 50s” or “average physical fitness age of 45” or the like, based on a year-by-year average value.

For example, in the above-described embodiment, an example is shown in which in addition to the exercise content information 5 to be proposed to the user of the elderly facility, such as, e.g., a day service facility, the graph 15 of the time series change of the physical fitness test data 3 and the graph 16 of the cognitive function test data 4 are displayed side by side, but the present invention is not limited thereto. For example, only the exercise content information 5 may be displayed. Further, for the physical fitness test data 3 and the cognitive function test data 4, only the most recent data may be displayed instead of the time series change.

For example, in the above-described embodiment, an example is shown in which in addition to the exercise content information 5 to be proposed to the user of the elderly facility, such as, e.g., a day service facility, the graph 15 of the time series change of the physical fitness test data 3 and the graph 16 of the cognitive function test data 4 are displayed side by side, but the present invention is not limited thereto. For example, the time series change may be displayed as a table instead of a graph. The time series change may include data of at least two different time points. The length of the period to be displayed as the time series change is not particularly limited.

For example, in the above-described embodiment, an example is shown in which the cognitive function test data 4 is screening test data by a blood test, but the present invention is not limited thereto. For example, the cognitive function test data 4 may be test data by a brain measurement, such as, e.g., an NIRS (near-infrared spectroscopy) a fMRI, and an EEG.

For example, in the above-described embodiment, an example is shown in which a physical fitness measurement is performed about once every three months, but the present invention is not limited thereto. For example, the physical fitness measurement may be increased once a month or more frequently. In addition, the frequency may be reduced in accordance with the physical condition of the user of the elderly facility, such as, e.g., a day service facility.

For example, in the above-described embodiment, an example is shown in which a screening test is performed about once every six months, but the present invention is not limited thereto. For example, the screening test may be increased to once a month or more frequently. Further, the frequency may be reduced in accordance with the state of the user of the elderly facility, such as, e.g., a day service facility.

For example, in the above-described embodiment, an example is shown in which the elderly facility, such as, e.g., a day service facility, is used, but the present invention is not limited thereto. For example, any institution or facility can be used, such as, e.g., a medical institution, an educational institution, and a commercial facility.

For example, in the above-described embodiment, an example is shown in which the facility staff performs data input, but the present invention is not limited thereto. For example, the input may be performed by the user of the elderly facility, such as, e.g., a day service facility, or the family of the user of the elderly facility, such as, e.g., a day service facility and medical personnel.

For example, in the above-described embodiment, an example is shown in which the exercise contents are proposed to the facility staff, but the present invention is not limited thereto. For example, the exercise contents may be proposed to the user of the day service facility, such as, e.g., a day service facility, or the family of the user, or the healthcare personnel.

### [Aspects]

It will be understood by those skilled in the art that the above-described exemplary embodiments are concrete examples of the following aspects.

### (Item 1)

A health risk reduction method comprising:
a step of acquiring physical fitness test data by a physical fitness measurement performed to measure physical fitness of a subject;
a step of acquiring cognitive function test data by a cognitive function test of the subject;
a step of acquiring exercise content information to be proposed to the subject, based on the physical fitness test data and the cognitive function test data; and
a step of outputting the exercise content information to be proposed to the subject.

(Item 2)

The health risk reduction method as recited in the above-described Item 1,
wherein the step of outputting the exercise content information includes a step of displaying the physical fitness test data and the cognitive function test data in addition to the exercise content information.

### (Item 3)

The health risk reduction method as recited in the above-described Item 1 or 2,
wherein, in the step of acquiring the exercise content information, the exercise content information for enhancing physical functions and the exercise content information for enhancing cognitive functions are acquired as the exercise content information.

### (Item 4)

The health risk reduction method as recited in the above-described Item 3,
wherein the step of acquiring the exercise content information includes:
a step of analyzing the physical fitness test data and the cognitive function test data; and
a step of selecting the exercise content information from a plurality of pieces of the exercise content information set in advance, based on an analysis result of the physical fitness test data and an analysis result of the cognitive function test data.

(Item 5)

The health risk reduction method according to the above-described Item 4,
wherein in the step of acquiring the exercise content information, a ratio of exercises for enhancing physical functions and a ratio of exercises for enhancing cognitive functions are differentiated, according to the analysis result of the physical fitness test data and the analysis result of the cognitive function test data.

(Item 6)

The health risk reduction method dependent on the above-described Item 2,
wherein the step of acquiring the physical fitness test data includes a step of storing the acquired physical fitness test data and a measurement time point in association with each other,
wherein the step of acquiring the cognitive function test data includes a step of storing the acquired cognitive function test data and the test time point in association with each other, and
wherein in the step of displaying the physical fitness test data and the cognitive function test data, the physical fitness test data and the cognitive function test data within a predetermined period are displayed along a time series.

(Item 7)

The health risk reduction method as recited in the above-described Item 6,
wherein in the step of displaying the physical fitness test data and the cognitive function test data, a time series change of the physical fitness test data within the predetermined period and a time series change of the cognitive function test data within the predetermined period are displayed.

(Item 8)

The health risk reduction method as recited in the above-described Item 7,
wherein in the step of displaying the physical fitness test data and the cognitive function test data, a graph of the time series change of the physical fitness test data within the predetermined period and a graph of the time series change of the cognitive function test data within the predetermined period are displayed side by side.

(Item 9)

The health risk reduction method as recited in the above-described Item 8,
wherein in the step of displaying the physical fitness test data and the cognitive function test data, the graph of the time series change of the physical fitness test data and the graph of the time series change of the cognitive function test data are displayed side by side, in addition to the exercise content information to be proposed to the subject.

(Item 10)

The health risk reduction method as recited in any one of the above-described Items 1 to 9,
wherein the exercise content information includes events and a number of times.

(Item 11)

A health risk reduction system comprising:
a physical fitness test data acquisition unit configured to acquire physical fitness test data by a physical fitness measurement performed to measure physical fitness of a subject;
a test data acquisition unit configured to acquire cognitive function test data by a cognitive function test of the subject;
an exercise content information acquisition unit configured to acquire exercise content information to be proposed to the subject, based on the physical fitness test data and the cognitive function test data; and
an output unit configured to output the exercise content information to be proposed to the subject.

(Item 12)

The health risk reduction program configured to make a computer
control to acquire physical fitness test data by a physical fitness measurement performed to measure physical fitness of a subject,
control to acquire cognitive function test data by a cognitive function test of the subject;
control to acquire exercise content information to be proposed to the subject, based on the physical fitness test data and the cognitive function test data; and
control to output the exercise content information to be proposed to the subject.

1. A health risk reduction method comprising:
a step of acquiring physical fitness test data by a physical fitness measurement performed to measure physical fitness of a subject;
a step of acquiring cognitive function test data by a cognitive function test of the subject;
a step of acquiring exercise content information to be proposed to the subject, based on the physical fitness test data and the cognitive function test data; and
a step of outputting the exercise content information to be proposed to the subject.

2. The health risk reduction method as recited in claim 1,
wherein the step of outputting the exercise content information includes a step of displaying the physical fitness test data and the cognitive function test data in addition to the exercise content information.

3. The health risk reduction method as recited in claim 1,
wherein, in the step of acquiring the exercise content information, the exercise content information for enhancing physical functions and the exercise content information for enhancing cognitive functions are acquired as the exercise content information.

4. The health risk reduction method as recited in claim 3,
wherein the step of acquiring the exercise content information includes:
a step of analyzing the physical fitness test data and the cognitive function test data; and
a step of selecting the exercise content information from a plurality of pieces of the exercise content information set in advance, based on an analysis result of the physical fitness test data and an analysis result of the cognitive function test data.

5. The health risk reduction method as recited in claim 4,
wherein in the step of acquiring the exercise content information, a ratio of exercises for enhancing physical functions and a ratio of exercises for enhancing cognitive functions are differentiated, according to the analysis result of the physical fitness test data and the analysis result of the cognitive function test data.

6. The health risk reduction method as recited in claim 2,
wherein the step of acquiring the physical fitness test data includes a step of storing the acquired physical fitness test data and a measurement time point in association with each other,
wherein the step of acquiring the cognitive function test data includes a step of storing the acquired cognitive function test data and the test time point in association with each other, and
wherein in the step of displaying the physical fitness test data and the cognitive function test data, the physical fitness test data and the cognitive function test data within a predetermined period are displayed along a time series.

7. The health risk reduction method as recited in claim 6,
wherein in the step of displaying the physical fitness test data and the cognitive function test data, a time series change of the physical fitness test data within the predetermined period and a time series change of the cognitive function test data within the predetermined period are displayed.

8. The health risk reduction method as recited in claim 7,
wherein in the step of displaying the physical fitness test data and the cognitive function test data, a graph of the time series change of the physical fitness test data within the predetermined period and a graph of the time series change of the cognitive function test data within the predetermined period are displayed side by side.

9. The health risk reduction method as recited in claim 8,
wherein in the step of displaying the physical fitness test data and the cognitive function test data, the graph of the time series change of the physical fitness test data and the graph of the time series change of the cognitive function test data are displayed side by side, in addition to the exercise content information to be proposed to the subject.

10. The health risk reduction method as recited in claim 1,
wherein the exercise content information includes events and a number of times.

11. A health risk reduction system comprising:
a physical fitness test data acquisition unit configured to acquire physical fitness test data by a physical fitness measurement performed to measure physical fitness of a subject;
a test data acquisition unit configured to acquire cognitive function test data by a cognitive function test of the subject;
an exercise content information acquisition unit configured to acquire exercise content information to be proposed to the subject, based on the physical fitness test data and the cognitive function test data; and
an output unit configured to output the exercise content information to be proposed to the subject.

12. A health risk reduction program configured to make a computer
control to acquire physical fitness test data by a physical fitness measurement performed to measure physical fitness of a subject,
control to acquire cognitive function test data by a cognitive function test of the subject;
control to acquire exercise content information to be proposed to the subject, based on the physical fitness test data and the cognitive function test data; and
control to output the exercise content information to be proposed to the subject.
